(19) United States
(12) Patent Application Publication
(10) Pub. No.: US 2021/0225505 A1
(43) Pub. Date: Jul. 22, 2021

(54) BIOLOGICAL DATA TRACKING SYSTEM AND METHOD

(71) Applicant: SPORTS DATA LABS, INC., Royal Oak, MI (US)

(72) Inventors: Vivek KHARE, Cupertino, CA (US); Stan MIMOTO, Bethel Island, CA (US); Mark GORSKI, Royal Oak, MI (US)

(73) Assignee: SPORTS DATA LABS, INC., Royal Oak, MI (US)

(21) Appl. No.: 17/054,277

(22) PCT Filed: Feb. 13, 2020
(86) PCT No.: PCT/US2020/018063

§ 371 Date: Nov. 10, 2020

Related U.S. Application Data
(63) Continuation-in-part of application No. 16/274,701, filed on Feb. 13, 2019.

(51) Int. Cl. G16H 40/67; A61B 5/00; G16H 50/20; G16H 50/30; G16H 40/40; G06F 16/957; H04L 29/08

(52) U.S. Cl. CPC G16H 40/67; G16H 20/17; A61B 5/0022; A61B 5/7475; G16H 50/20; G16H 50/30; G16H 40/40; G06F 16/9577; H04L 67/02; A61B 2562/0219; A61B 2560/0242; A61B 2503/10; A61B 5/681; A61B 2503/40; A61B 5/742

(57) ABSTRACT

A browser-based biological tracking system connects a computing device to a set of wireless communication-enabled sensors. The system includes a connection application that is automatically downloaded from a server in response to a user accessing a webpage. The connection application sends data streams from the sensors to the server. The server calculates readings which are then displayed in a webpage. The set of sensors may include multiple types of sensors which utilize different communication interfaces. Some readings, sometimes referred to as aggregate insights, are computed based on measurements from multiple sensors.

### TECHNICAL FIELD

In at least one aspect, the present disclosure relates to the field of monitoring systems utilizing data from sensors applied to living beings. More particularly, the disclosure pertains to a system for configuring and collecting data directly from one or more wearable sensors using a connection application that is downloadable and executable via a web browser.

### BACKGROUND

A number of physiological data sensors have been developed to assist subjects such as athletes during training or competition. Some of these sensors are wearable or fixed to the subject's skin to measure various metrics and wirelessly send the measurements to a device such as a cell phone or tablet for display. Typically, each sensor is associated with an application program which a user must download and install on the cell phone or tablet. Each application receives and displays data from a single sensor. Some of the application programs may be capable of uploading data to a server for later processing.

If there are multiple persons or one person using multiple sensors, multiple devices (e.g., cell phones or tablets) and/or multiple applications may be required to operate the one or more sensors and to display the data. Any computation that relies on data from multiple sensors must rely on the data being uploaded to a server. Even then, it may be difficult to correlate the data from one sensor with the data from another sensor.

### SUMMARY OF THE DISCLOSURE

In at least one aspect, a data tracking system, and in particular, a biological data tracking system, includes one or more wireless communication-enabled data sensors (e.g., biological data sensors), a computing device, and a connection application. The one or more wireless communication-enabled data sensors may include at least two different types of sensors which may communicate with the connection application using different communication interfaces. The computing device includes a network connection (e.g., internet) and browser application (e.g., executing browser software). The connection application, which executes within the browser, is configured (e.g., programmed) to establish one or more wireless communication links with each of the one or more sensors, receive one or more streams of data from the one or more sensors, and display, via the browser application, one or more readings derived from at least a portion of the streamed data. The connection application may also transmit one or more commands to the one or more wireless communication-enabled data sensors to change, adjust, and/or modify one or more sensor settings. At least one of the readings may be derived from at least a portion of the one or more data streams from two or more different sensors. The system may also include a server configured to receive the one or more streams of data via a network connection (e.g., internet) and compute the readings. The server may also transmit the connection application program to the browser in response to a user accessing a web page.

In another aspect, a data tracking system, and in particular, a biological data tracking system, includes a connection application and a server. The connection application establishes wireless communication with each of a plurality of wireless communication-enabled data sensors (e.g., wearable biological sensors and/or other wireless communication-enabled data sensors), receives one or more streams of data from the sensors, and displays, via a browser application, one or more readings derived from at least a portion of the streamed data. The server may transmit the connection application to the browser in response to a user accessing a web page. The connection application may transmit the one or more streams of data to the server which then calculates one or more readings. The plurality of wireless communication-enabled sensors may include at least two different types of sensors, including sensors that communicate with the connection application using different communication interfaces. At least one of the readings may be derived from at least a portion of the data streams from two different sensors. The connection application may also transmit one or more commands to a remote-controlled device and/or a subset of the plurality of sensors to change one or more sensor settings, which may occur in sequence or simultaneously.

In another aspect, a method of tracking biological data includes transmitting a connection application from a server to a browser, detecting the at least one wireless communication-enabled sensor, wirelessly receiving one or more data streams from the at least one sensor, and displaying one or more readings. The server transmits the application to the browser in response to a user accessing a web page. The connection application detects the one or more sensors and directly receives at least a portion of the one or more data streams. The one or more readings are derived from at least a portion of the one or more data streams and displayed, at least in part, in the browser. The method may also include transmitting at least a portion of the one or more data streams from the browser to the server and transmitting the one or more readings from the server to the browser. The connection application may send one or more commands to the one or more sensors to change one or more sensor settings. The one or more sensors may include at least two types of sensors which may transmit one or more data streams to the connection application using two different communication interfaces.

### BRIEF DESCRIPTION OF THE DRAWINGS

FIG. 1 is a schematic diagram of a biological data tracking system.

FIG. 2 is a block diagram illustrating the state of a computing system before a user launches the data tracking system webpage.

FIG. 3 is a flowchart for using the biological data tracking system of FIG. 1.

FIG. 4 is a block diagram illustrating the state of the system after an intermediate step in the method of FIG. 3.

FIG. 5 is an exemplary display page for selecting sensors.

FIG. 6 is an exemplary data entry screen for assigning sensors to subjects and for setting sensor parameters.

FIG. 7 is a block diagram illustrating the state of the system during an operation phase.

FIG. 8 is an exemplary data entry screen for selecting readings for display.

FIG. 9 is an exemplary display page for readings.

FIG. 10 is an exemplary display page for readings featuring an integrated video-based system.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present invention. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “server” refers to any computer, computing device, mobile phone, desktop computer, notebook computer or laptop computer, stationary computer, a wearable computing system, distributed system, blade, gateway, switch, processing device, or combination thereof adapted to perform the methods and functions set forth herein.

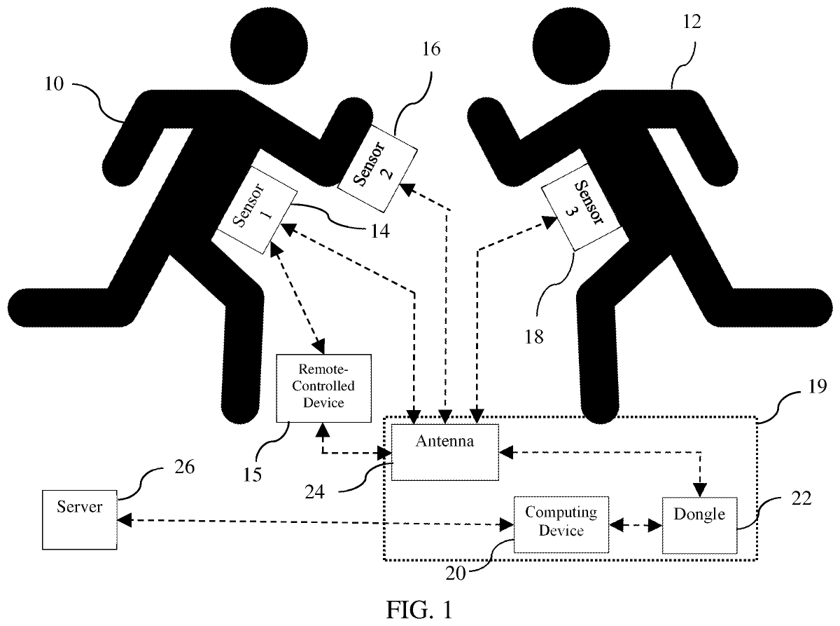

FIG. 1 illustrates a system to track and analyze biological data from one or more subjects in real-time or near real-time via a web browser-based system. A subject is a human or other animal, including birds and fish, as well as all mammals including primates (particularly higher primates), horses, sheep, dogs, rodents, guinea pigs, pigs, cats, whales, rabbits, and cows. The subjects may be, for example, humans participating in athletic training or competition, playing a video game, monitoring their personal health, sleeping, providing their data to a third party, participating in a research or clinical study, or participating in a fitness class. A subject can also be a derivative of a human or other animal (e.g., lab-generated organism derived at least in part from a human or other animal), one or more individual components of a human or other animal that make up the human or other animal (e.g., cells, proteins, amino acid sequences, tissues, hairs, limbs), and one or more artificial creations that share one or more characteristics with a human or other animal (e.g., lab-grown human brain cells that produce an electrical signal similar to that of human brain cells). In a refinement, the subject may be a machine programmable by one or more computing systems (e.g., robot, autonomous vehicle, mechanical arm) that shares at least one common characteristic with a human or other animal and from which one or more types of biological data can be derived, which may be, at least in part, artificial in nature but shares at least one common characteristic to real biological data, or a derivative thereof.

Two such subjects, 10 and 12, are shown in FIG. 1. The actual number of subjects may vary from one to several dozen or even more. Each subject is monitored by at least one wireless communication-enabled sensor 14, 16, and 18. In some cases, “sensor” is referred to as “data sensor,” and therefore these terms are equivalent as used herein. Wireless communication-enabled sensors include both wearable sensors, which may be biological in nature, and biological sensors, which may be wearable in nature. In some cases, “wearable sensor” and “biological sensor” are also interchangeable, and therefore the use of one term should be interpreted as including the other, and vice versa. Wireless communication-enabled sensors include sensors and/or its one or more appendices that are attached directly to the subject including the subject's skin, sensors that are embedded under the subject's skin, and sensors that are attached to the subject's clothing. Wireless communication-enabled sensors also include sensors and/or its one or more appendices that are affixed to, are in contact with, or send an electronic communication in relation to or derived from, the subject including a subject's skin, eyeball, vital organ, muscle, hair, veins, blood, blood vessels, tissue, or skeletal system, embedded in a subject, lodged or implanted in a subject, integrated to comprise at least a portion of the subject, or ingested by a subject. For example, a saliva sensor affixed to a tooth, a set of teeth, or an apparatus that is in contact with one or more teeth, a sensor that extracts DNA information derived from a subject's hair, a sensor affixed to or implanted in the subject's brain that may detect brain signals from neurons, a sensor that is ingested by an individual to track one or more biological functions, or a sensor attached to, or integrated with, a machine (e.g., robot) that shares at least one common characteristic with a subject (e.g., a robotic arm with an ability to perform one or more tasks similar to that of a human; a robot with an ability to process information similar to that of a human). Advantageously, the machine itself may be comprised of one or more sensors, and may be classified as both a sensor and a subject. In a refinement, the one or more sensors and/or its one or more appendices are integrated into or as part of, affixed to or embedded within, a textile, fabric, cloth, material, fixture, object, or apparatus that contacts or is in communication with a targeted subject either directly or via one or more intermediaries. Examples include a sensor attached to the skin via an adhesive, a sensor integrated or embedded into a shirt, a sensor integrated or embedded into a steering wheel, a sensor integrated or embedded into a video game controller, a sensor integrated or embedded into a basketball that is in contact with the subject's hands, a sensor integrated or embedded into a hockey stick or a hockey puck that is in intermittent contact with an intermediary being held by the subject (e.g., hockey stick), a sensor integrated or embedded into the one or more handles or grips of a fitness machine (e.g., treadmill, bicycle, bench press), a sensor that is integrated within a robot (e.g., robotic arm), and a sensor integrated or embedded into a shoe that may contact the targeted subject through the intermediary sock and adhesive tape wrapped around the targeted subject's ankle. In another refinement, sensors may be interwoven into, embedded into, integrated with, or affixed to, a flooring or within the ground (e.g., artificial turf grass, basketball floor, soccer field), a seat/chair, helmet, or an object that is in contact with the subject either directly or via one or more intermediaries (e.g., a subject is in contact with a sensor in a seat via a clothing interstitial). In another refinement, the sensor and/or its one or more appendices may be in contact with a particle or object derived of the subject's body (e.g., tissue from an organ, hair from the subject) from which the one or more sensors derive or provide information that can be calculated or converted into biological data. Advantageously, a wireless communication-enabled sensor may have multiple sensors within a single sensor. For example, an ECG sensor may also have a gyroscope, accelerometer, and magnetometer capable of capturing and providing XYZ coordinates.

Various types of wireless communication-enabled sensors are programmed to detect various types of data including biological data. Wireless communication-enabled sensors include biological sensors (biosensors) that collect biosignals which in the context of the present embodiment are any signals or properties in, or derived from, animals or other subjects that can be continually or intermittently measured, monitored, observed, calculated, computed, or interpreted, including both electrical and non-electrical signals, optical measurements, and artificially-generated information. A biological sensor can gather physiological, biometric, chemical, biomechanical, genetic, genomic, location or other biological data from one or more targeted subjects. For example, some biological sensors may measure, or provide information that can be converted into or derived from, physiological metrics such as eye-tracking, blood pressure, electrocardiogram signals, blood flow, body temperature, perspiration levels, biochemical structure, pulse, oxygenation, respiratory rate, blood analysis, biological fluid, biochemical composition, glucose levels, hydration levels, lactate levels, sodium levels, potassium levels, EEG, EMG, EOG, heart rate, neurological-related information, genetic-related information, genomic-related information, muscle activity, or a combination thereof. Some biological sensors may detect biological data such as biomechanical metrics, which may include, for example, angular velocity, joint paths, or position or accelerations in various directions from which a subject's movements may be characterized. Some biological sensors may capture biological data such as location coordinates and positional data (e.g., from GPS, RFID sensors), facial recognition data, skeletal data, kinesthetic data (e.g., physical pressure captured from a sensor located at the bottom of a shoe), or auditory data related to the one or more target individuals. Some biological sensors are image-based and collect, provide and/or analyze video or other visual data or information (e.g., still or moving images, including video, MRIs, computed tomography scans, ultrasounds, X-rays) upon which biological data can be detected, extrapolated, observed, calculated, or computed (e.g., biomechanical movements, location, a fracture based on an X-Ray, or stress or a disease based on video or image-based visual analysis of a subject). In addition to biological data related to one or more targeted subjects, some biosensors may measure ambient temperature and humidity, elevation, and barometric pressure. In a refinement, one or more sensors provide biological data that include one or more calculations, computations, predictions, estimations, inferences, observations, or forecasts that are derived, at least in part, from the captured sensor data.

In the embodiment of FIG. 1, a remote-controlled device 15 may also be utilized. Typically, a remote-controlled device is an electronic device capable of being controlled by another device via one or more communication links that may occur wirelessly. The remote-controlled device may be wearable by the subject, attached to a subject, embedded within a subject, lodged or implanted in a subject, or used as a standalone device. Examples of remote-controlled devices include wearable insulin pumps, heart beat regulation devices, and the like. Advantageously, the one or more remote-controlled devices may be paired with, attached to, integrated or embedded into, in contact with, or housed within one or more of the same units with, one or more wireless communication-enabled sensors, which may aid in one or more biological functions. In a refinement, the remote-controlled device may be classified as a wireless communication-enabled sensor and include one or more sensing capabilities (e.g., a remotely controlled intravenous injection and transfusion system with a flow control valve sensor, or an insulin pump that includes a glucose sensor to monitor glucose levels, or a wearable or implantable defibrillator with heart rate tracking capabilities). In another refinement, a remote-controlled device may be utilized with other remote-controlled devices to aid one or more biological functions.

Due to the mobility of the subjects and/or other considerations, wired connections to transmit data from the sensors are often not feasible. Therefore, the sensors communicate with the other parts of the system wirelessly, for example using Bluetooth Low Energy (BLE) technology, Wifi, Ant+, LoRa, Zigbee, cellular networks, and the like. However, the present invention is not limited by the technologies that sensors use to transmit and/or receive signals. The transmission of wireless signals from the one or more sensors can occur via a transmission subsystem 19, which may include one or more receivers, transmitters, transceivers, and/or supporting components (e.g., dongle) that utilize a single antenna or multiple antennas (e.g., which may be configured as part of a mesh network). The transmission subsystem and/or its one or more components may be housed within the computing device or may be external to the computing device (e.g., a dongle connected to the computing device which is comprised of one or more hardware and/or software components that facilitates wireless communication and is part of the transmission subsystem 19). In a refinement, transmission subsystem 19 and/or its one or more of its components are integral to the one or more sensors.

In the embodiment of FIG. 1, the one or more sensors communicate with computing device 20 via a BlueTooth dongle 22 and an antenna 24. An external dongle (e.g., BlueTooth dongle, WiFi dongle) may be utilized in the event the transmission capabilities of the computing device necessitate external support (e.g., to extend the range of the transmission communication between the one or more sensors and computing device). Bluetooth dongle 22, in conjunction with the antenna 24, act as a single unit to amplify or “boost” the signal strength, quality, and speed of the transmission signal (e.g., to ensure there is minimal data packet loss), as well as enable utilization of the one or more sensors (e.g., connection with a plurality of sensors). These components may be separate or housed within a single unit. However, a dongle may not be required depending on the computing system and its capabilities. Computing device 20 may be, for example, a personal computer, laptop computer, or a stationary computing system. However, computing device 20 can be any computing device, including wearable computing systems such as smart glasses or headsets that contain a display. The term “computing device” refers generally to any device that can perform at least one function, including communicating with another computing device. In a refinement, a computing device includes a central processing unit that can execute program steps and memory for storing data and program code. Advantageously, the one or more sensors, the computing device, and/or the transmission subsystem 19 may be housed within, or attached to, the same unit (e.g., smart glasses with biological sensors embedded in the glasses). As previously mentioned, the components that would comprise a signal-amplifying transmitter/receiver like a dongle and/or an antenna may also be integrated as part of the computing device.

Antenna 24 may be a directional or omnidirectional antenna positioned to provide high gain with respect to the area in which the subjects move and low gain with respect to other areas. The antenna may be integrated with the computing device or a separate unit. While different antennas may have different advantages in different environments, a carefully selected and positioned directional antenna may be optimal and provide multiple advantages over a non-directional antenna. First, the higher gain in the areas occupied by the subjects increases the transmission range. Secondly, especially in spectator sports or other crowded environments, there may be a high number of signals in the sane frequency range from surrounding areas that could cause interference. Many spectators have mobile phones that communicate with other devices via one or more transmission protocols (e.g., Bluetooth, WiFi) which use the same frequency. An antenna with low gain toward these areas dramatically reduces the likelihood of interference. However, one or more types of antennas may be utilized including wire antennas (e.g., short dipole antenna, dipole antenna, half-wave dipole, broadband dipoles, monopole antenna, folded dipole antenna, loop antenna, cloverleaf antenna), traveling wave antennas (e.g., helical antennas, yagi antennas, spiral antennas), reflector antennas (e.g., corner reflector, parabolic reflector/dish antenna), microstrip antennas (e.g., rectangular microstrip (patch) antennas, planar inverted-f antennas), log-periodic antennas (e.g., bow-tic antennas, log-periodic antennas, log-periodic dipole array), aperture antennas (e.g., slot antenna, cavity-backed slot antenna, inverted-f antenna, slotted waveguide antenna, horn antenna, Vivaldi antenna), NFC antennas, fractal antennas, and the like, which may be comprised of one or more different materials and may take one or more different shapes or form factors (e.g., flexible antenna as part of a watch strap). However, the present invention is not limited by the materials or technologies that sensors use to transmit and receive signals.

In a refinement, the one or more transmission subsystems, or components of the transmission subsystem such as an antenna and/or dongle, may be wearable and may be affixed to, in contact with, or integrated with, the subject either directly or via one or more intermediaries (e.g., clothing). In another refinement, the one or more antennas or transmission subsystems include an on or in-body transceiver (“on-body transceiver”) that optionally acts as another sensor or is integrated within a biological sensor. The on-body transceiver is operable to communicate with the one or more wireless communication-enabled sensors on a targeted subject's body or across one or more target subjects, and may itself track one or more types of biological data (e.g., positional data). In a refinement, the on-body transceiver is affixed to, integrated with, or in contact with, a subject's skin, hair, vital organ, muscle, skeletal system, eyeball, clothing, object, or other apparatus on the subject. Advantageously, the on-body transceiver collects data in real-time or near real-time from one or more sensors on a subject's body, communicating with each sensor using the one or more transmission protocols of that particular sensor. The on-body transceiver may also act as a data collection hub. In a variation, transmission subsystem 19 includes an aerial transceiver for continuous or intermittent streaming from sensors on moving bodies or objects. Examples of aerial transceivers include, but are not limited to, communications satellites, drones, and unnamed aerial vehicles with attached transceivers. Additional details of unmanned aerial vehicle-based data collection and distribution systems are disclosed in U.S. patent Ser. No. 16/517,012 filed Jul. 19, 2019; the entire disclosure of which is hereby incorporated by reference. In another variation, transmission subsystem 19 includes a transceiver embedded or integrated as part of a floor or ground (including a field), with transmission occurring via direct contact with a surface (e.g., in the event the sensor is located on or near the bottom of the shoe). Finally, computing device 20 is connected to a server 26 by a network, which may include some combination of Wifi and hardwired internet connections.

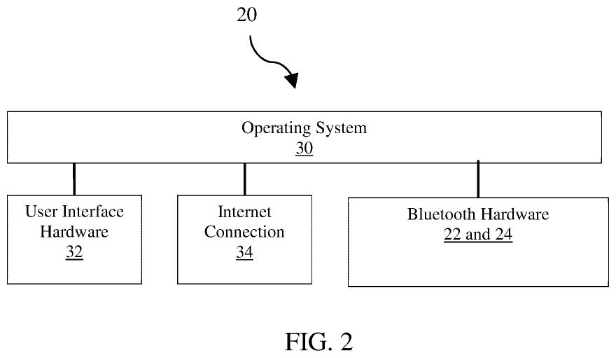

FIG. 2 illustrates the status of the system at the beginning of a data monitoring session. Computing device 20 includes an operating system 30 that coordinates interactions between various types of hardware and software. The hardware present for this illustration includes BlueTooth hardware such as the dongle 22 and antenna 24 illustrated in FIG. 1. It also includes various user interface hardware 32 such as a keyboard, a pointing device such as a mouse (or a feature similar to a pointing device that enables an action to be taken within the browser, e.g., voice-controlled action via a virtual assistant; eye-tracking within spatial computing systems that enables an eye-controlled action), and a display device. In a refinement, a gesture controller that enables hand or body movements to indicate an action may be utilized for keyboard and mouse functions. Typically, a display device communicates information in visual form. A display may include a plurality of displays that comprise the display. Advantageously, the display may communicate information utilizing one or more other mechanisms including via an audio or aural format (e.g., verbal communication of biological readings), via a physical gesture (e.g., a physical vibration which provides information related to the one or more biological readings), or a combination thereof. The display device may take one or more forms. Examples of where one or more readings may be displayed include via one or more monitors, a smartwatch, or within smart glasses or eyewear where the readings can be visualized. Advantageously, the one or more sensors, computing device, transmission subsystem, server, and display device, or a combination thereof, may be housed within or attached to, the same unit. As also illustrated in FIG. 2, the computing device also has a network connection 34, such as the internet, which may include both hardware and software aspects, or pre-loaded hardware and software aspects that do not necessitate an internet connection.

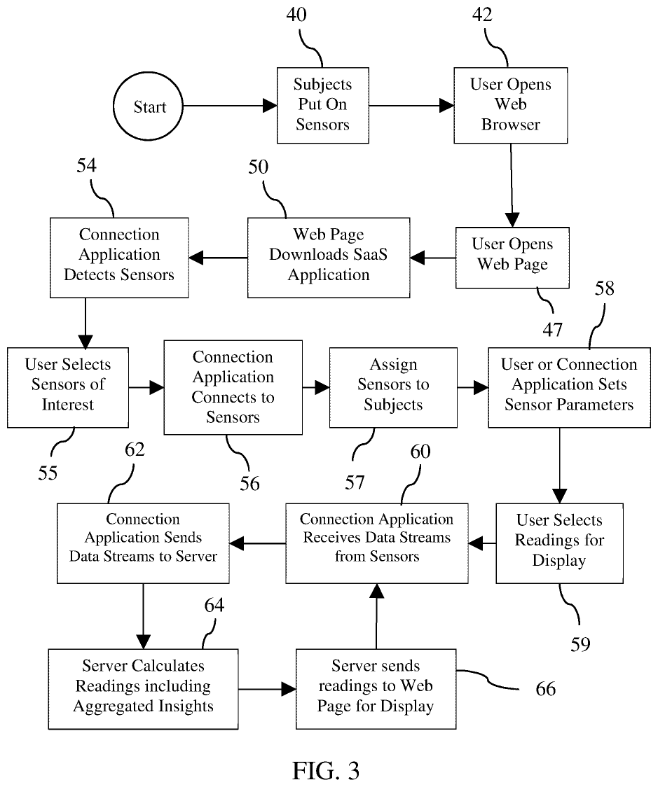

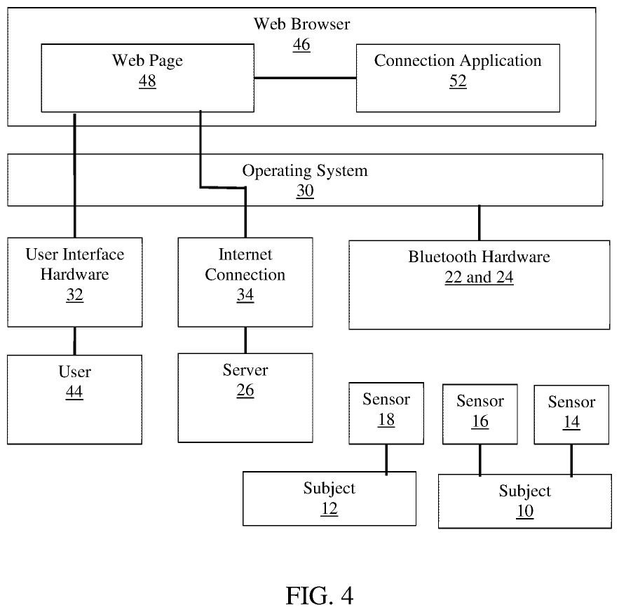

FIG. 3 is a flow chart for a typical operational sequence of utilizing the system of FIG. 1. At 40, the subjects put on the one or more sensors and power them on if necessary. FIG. 4 illustrates the status of the system following step 40. Note that no connection yet exists between the one or more sensors and the computing device. At 42 of FIG. 3, a user 44 (FIG. 4), interacting with the user interface hardware 32, opens a web browser 46. A web browser is a general-purpose software application that is commonly found on computing devices including personal computers. A user may start the web browser by, for example, clicking on an icon with a pointing device such as a mouse, verbally communicating the command using a voice-activated assistant, communicating the command with a physical gesture (e.g., finger swipe or eye movement), or neurologically communicating the command (e.g., a computing device like a brain-computer interface may acquire one or more of the subject's brain signals from neurons, analyze the one or more brain signals, and translate the one or more brain signals into commands that are relayed to an output device to carry out a desired action. Acquisition of brain signals may occur via a number of different mechanisms including one or more sensors that may be implanted into the subject's brain). The web browser starts by displaying a default page such as a search engine. At 47, the user opens web page 48 which is associated with the tracking system of FIG. 1. The user may open this page by providing a Uniform Resource Locator (URL) via typing or other mechanism (e.g., verbal command), by using a search engine, or by selecting a saved entry from a previous session.

At 50, the web page 48 triggers downloading of web application 52 (FIG. 4) to the web browser. FIG. 4 illustrates the state of the system following step 50. A web page is a software entity that conveys information, usually from a server, to the user and collects information and one or more commands from the user to be conveyed to the server. A data connection application 52, on the other hand, performs non-trivial computation and may be programmed to interact with hardware besides the user interface hardware 32 and the internet connection 34. Advantageously, connection application 52 may be a Software as a Service (SaaS) application, meaning that, from the user's perspective, the functionality is being provided as a service on the internet. The user does not direct and does not need to be aware of, any downloading or installation of software to his or her computer. Connection application 52 may also be considered a communication and control application given its wide range of functionalities related to connecting with, communicating with, and controlling one or more sensors.

In FIG. 3, at 54, the connection application 52 utilizes the transmission hardware 22 and 24 to detect the presence of sensors 14, 16, and 18 and instructs web page 48 to display a list of available sensors to the user. Advantageously, the connection application may communicate, either continuously, intermittently, or on scheduled intervals, with the computing device and/or the server to send out and receive signals to determine whether any new sensors are available for integration and/or pairing with the connection application. In a refinement, the connection application can establish wireless connection links with the sensors and/or wired connection links with the sensors (i.e., wired sensors). At 55, the user selects the sensors of interest, for example, by clicking on one or more buttons displayed by web page 48 or providing a command (e.g., verbal, physical gesture). In a refinement, the user may utilize an RFID-enabled tag, facial or voice recognition systems, or similar functioning offerings to identify and associate the one or more sensors with the one or more subjects, or associate the combined offering, within the browser-based system. In another refinement, the connection application may communicate with the one or more sensors that are already assigned to any given subject to determine whether the user or administrator of the connection application wants to pair with the one or more subjects. For example, in a group fitness class, each subject may have their own sensor or multiple sensors that have been previously assigned to the one or more subjects. The administrator of the fitness class may be able to detect the sensor through the connection application for each subject and automatically assign the one or more subjects to the fitness class activity. The sensor detection and corresponding association with the subject through the connection application may also provide immediate access to the subject's profile and enable the one or more sensors to stream automatically to that subject's specific profile within the browser-based application. Advantageously, such a system would enable efficient recognition of a subject and their one or more sensors across multiple computing devices and/or across multiple browsers should the subject desire the data to be streamed, for example, in multiple locations.

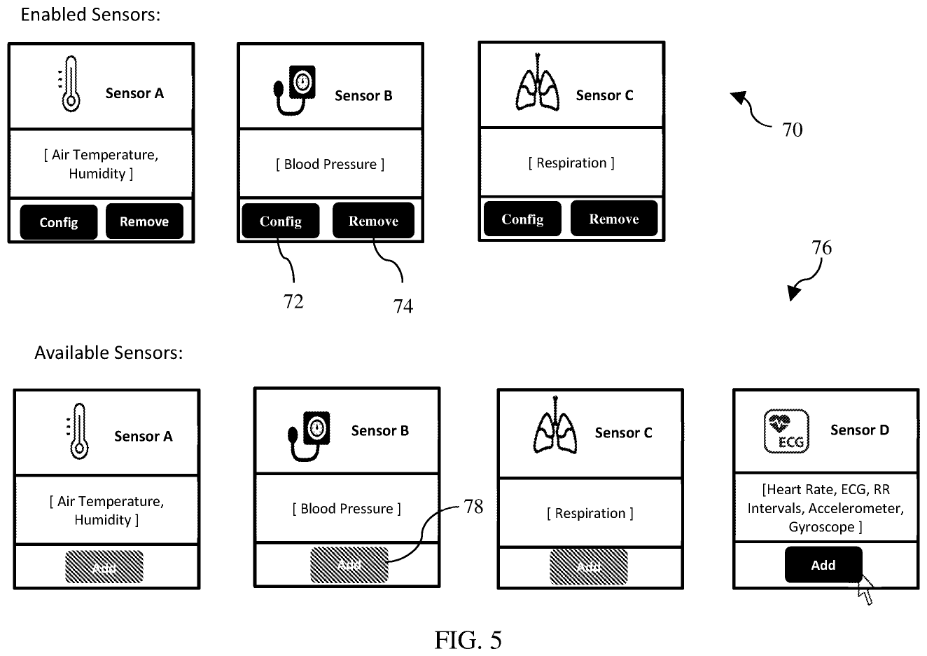

FIG. 5 illustrates an exemplary webpage 48 for selecting one or more sensors and setting one or more sensor parameters/functionalities. Sensors that have already been selected are displayed in a first area of the screen 70. For each such sensor, the display indicates, for example, the type of sensor, the quantities which the sensor is capable of measuring, and one or more buttons. Button 72 permits the user to configure the sensor as discussed below. In the context of sensors, tasks related to the term “configure” include any ability to control, program, change, adjust, and/or modify any parameter or functionality of the sensor. Button 74 permits the user to un-select the sensor. Sensors that have been detected but not yet selected are displayed in a second area of the screen 76. For each such sensor, the display may indicate, for example, the type of sensor, the quantities which the sensor is capable of measuring, and one button. Button 78 permits the user to select the sensor, after which it is moved to area 70. In a refinement, one or more buttons may be replaced by one or more verbal, neurological, physical, or other communication cues provided to the software application that perform one or more commands (e.g., verbally telling the software application via a virtual assistant to perform a command, such as selecting or unselecting a specific sensor).

In FIG. 3, at 56, the connection application connects to the selected one or more data sensors. At 57, the user assigns one or more particular sensors to one or more particular subjects. The system may provide a default value to simplify this process when only one subject is involved. At 58, the connection application is capable of sending one or more commands to the one or more sensors to set one or more sensor parameters and start streaming measurements. More specifically, the connection application is configured (programmed) to transmit one or more commands to a remote-controlled device and/or to the one or more wireless communication-enabled data sensors to change, adjust, and/or modify one or more sensor settings. The tem “configured” is inclusive of the connection application being operational and programmable (e.g., an ability to be programmed) to do so. A sensor setting includes any parameter or functionality of the sensor. A change, adjustment, or modification of a sensor setting includes any control and/or configuring of a sensor and/or its one or more settings. One or more settings may include items like mode of operation, sampling rate, data range, gain, power mode, voltages, etc. It may also include an ability to provide updates to the sensor (e.g., firmware), or command the sensor to take an action. In a refinement, one or more commands act to change, adjust, and/or modify administration of one or more substances (e.g., cause the remote-controlled device to perform these actions). Substances can include drugs, prescriptions, medications, or any physical matter or material. Administration includes strength, quantity, dosage, timing, and frequency of substances, as well as the actual release, dispense, and application of any given substance. For example, a patient with diabetes may wear one or more sensors that transmit one or more glucose readings to the connection application, as well as a remote-controlled device such as an insulin pump to aid the pancreas in a biological function of providing insulin to the body. Based on the one or more readings from the one or more glucose sensors, the connection application may be programmed to send one or more commands to the one or more sensors and/or remote-controlled devices (if they are separate or not paired in any way) worn by the patient to change, adjust, and/or modify a setting (e.g., release insulin into the patient's body). The command sent by the connection application to the sensor and/or the device (if separate) may be, for example, to increase the sampling rate of the number of readings from the glucose sensor, or adjust the amount of insulin administered to the patient and release the insulin into the patient's body. The command sent by the connection application may be programmed to be sent automatically based on one or more predefined thresholds (e.g., if the glucose levels are too high, the pump or combined pump/glucose sensor releases insulin, which may be adjusted based on the specific glucose level of the patient). In a variation, the insulin pump may be paired with a glucose sensor to monitor and regulate one or more glucose-related biological functions (e.g., blood sugar levels) based upon predefined thresholds communicated by the connection application. In a refinement, the amount of insulin administered to the patient may be adjusted by another one or more users (e.g., doctor or medical professional) who are also accessing the connection application and, in turn, send one or more commands to the connection application to change a sensor setting (in this case, the amount of insulin being released in the patient's body via the insulin device or insulin sensor based on the glucose reading). Advantageously, adjustments may be made on a separate computing device from the patient. Administration may be determined, at least in part, via one or more calculations, computations, predictions, estimations, inferences, observations, or forecasts that that utilize at least a portion of one or more data readings and/or its one or more derivatives.

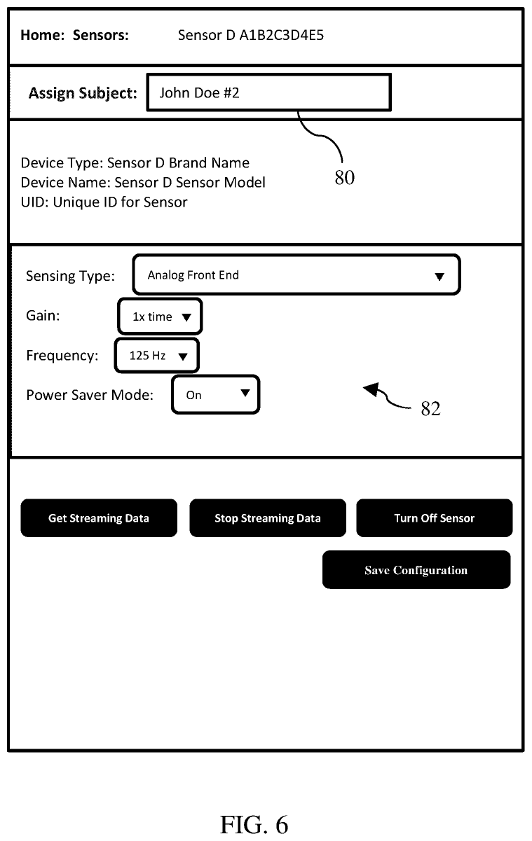

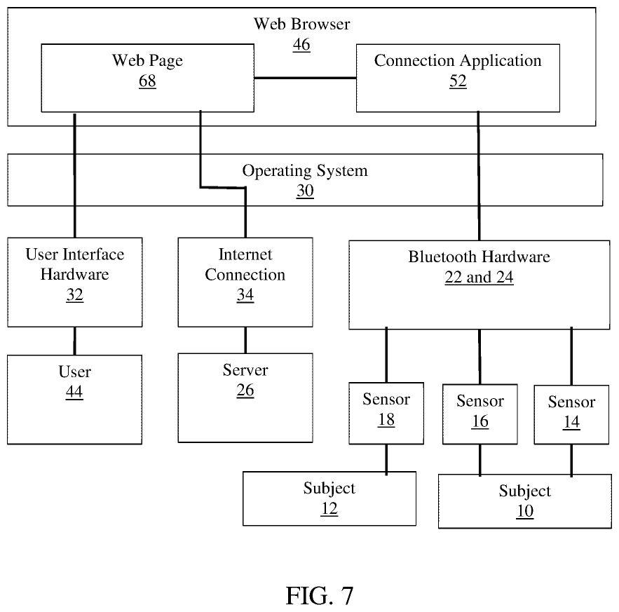

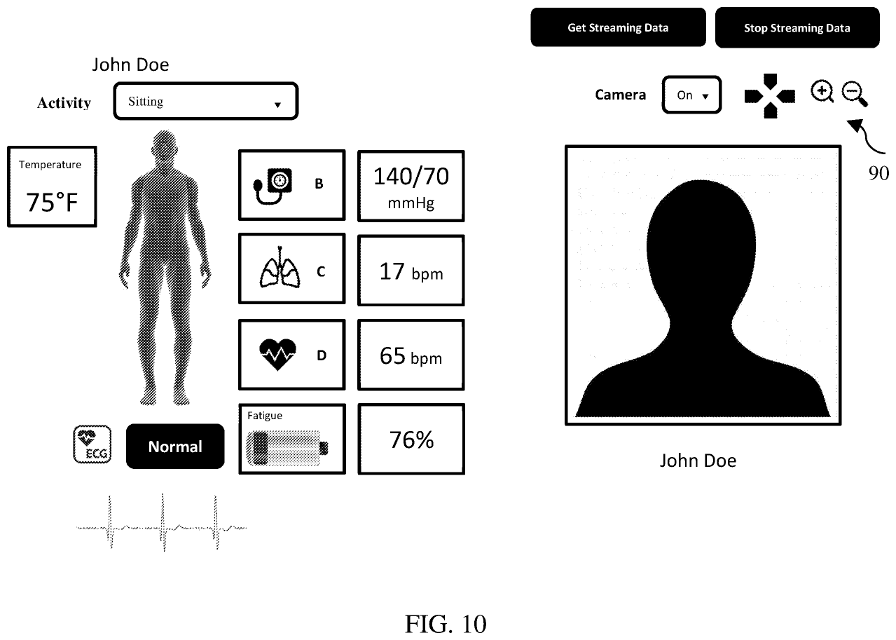

The user may be presented with the ability to edit the one or more parameter settings through a data entry screen such as that illustrated in FIG. 6. Text entry box 80 permits the user to assign the sensor to a particular subject. Selection boxes in area 82 allow the user to set one or more sensor parameters. In some cases, the change, adjustment, and/or modification of one or more settings may occur while the data is streaming. For example, in the case of a camera sensor providing a video feed of observed biological data such as a skin irritation on a patient, the setting changed and action taken may be for the camera to zoom in on the area of the irritation. FIG. 10 at 90 demonstrates a user's ability to navigate and control a camera functionality. The camera sensor may be integrated within the computing device, attached to the computing device, or separate from the computing device. Advantageously, this action may be taken by the user utilizing the one or more sensors on the computing device directly receiving the one or more streamed readings, or by one or more third parties on one or more computing devices that are not directly receiving the one or more streamed readings but are monitoring the user of the connection application. The state of the system after selecting sensors and assigning them to subjects is illustrated in FIG. 7.

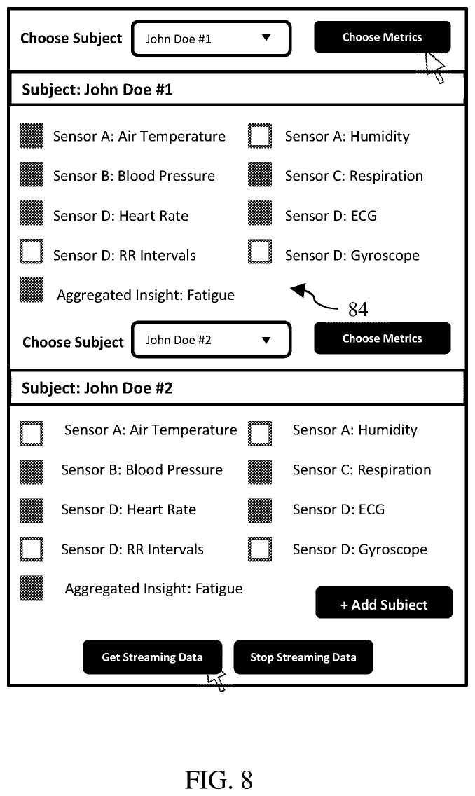

In FIG. 3, at 59, the user selects the one or more readings for display for each subject. This may be performed via a data entry screen such as that illustrated in FIG. 8. A checkbox or other indicator is displayed for each measurement that is available. In a refinement, each available measurement may be communicated to the user verbally, and the one or more indicators to select each available measurement may be initiated by a verbal, physical, or neurological gesture from the user. Note that the number of measurements available may exceed the number of selected sensors, as some sensors are capable of measuring multiple quanities. In a refinement, in the event the one or more sensors are capable of capturing a plurality of metrics (e.g., ECG, respiratory rate, muscle activity, temperature, galvanic skin response) where the sensor, environment, or other condition does not allow for the collection of multiple metrics simultaneously from that particular sensor, the connection application may be programmed to change, adjust, or modify the one or more readings captured, as well as when the one or readings are captured (e.g., intervals, intermittently, continuously). This may be dependent on one or more factors including the subject's activity.

Some readings may be derived from multiple data streams that come from different biological sensors. Such readings may be referred to as aggregated insights. One or more aggregated insights may be derived from data streams from different types of sensors. For example, the fatigue reading 84 may be derived from a combination of heart rate (e.g., from an electrocardiogram sensor), blood pressure, ambient temperature, etc. As another example, an athlete's reaction time may be derived by comparing a sensor-derived acceleration measurement from the first athlete to an acceleration measurement from a second athlete. The ability to gather these different data streams into a single application greatly facilitates one or more computations, as well as speed of the one or more computations, of these aggregated insights. If the data streams are collected by separate connection applications and then transmitted to the server, the latency of transmission may preclude real-time or near real-time display. Near real-time means low enough latency for the subject to react to the reading during the activity. Additionally, variability of latency between sensors may lead to the data streams not being properly synchronized as they arrive at the server.

Some readings may be derived from, at least in part, the streamed data, and include other types of information that are incorporated in the readings. For example, a reading may factor in non-streaming data such as a subject's age, height, social habits, medical history, personal history, and other inputs that may impact one or more readings. In another example, an athlete may have readings derived from the streamed data that incorporates one or more statistics (e.g., in basketball, points scored, rebounds, assists, minutes played). In a refinement, one or more aggregated insights may be derived from two or more data streams within the same sensor. For example, energy expenditure of a subject can be calculated by taking into account the maximal oxygen consumption readings (VO2 max) and the heart rate readings of a subject, which may be derived from the same sensor. In this example, consumption readings (VO2 max) and the heart rate readings may also be derived from two separate sensors. In another refinement, one or more aggregated insights may be derived from a combination of one or more data streams derived from one or more sensors and one or more non-sensor data streams. For example, another way to calculate energy expenditure includes utilizing heart rate measurements along with data that may not be derived from a sensor including metabolic equivalents (METs) for any given activity, age, weight, and/or duration of an activity.

Advantageously, one or more aggregated insights may be derived from two or more data streams, at least one of which is artificially generated. In a refinement, the one or more aggregated insights may be artificially generated and may occur via running one or more simulations that utilize at least a portion of the one or more data streams and/or its one or more derivatives derived from one or more sensors. In another refinement, at least a portion of the one or more readings and/or its one or more derivatives are generated or adjusted, at least in part, by one or more artificial intelligence or machine learning techniques (e.g., including via one or more trained neural networks). The term “generated” can include any creation, calculation, computation, or adjustment (e.g., modification) made to one or more data sets. For example, a biological sensor may produce data that includes noise artifacts due to the subject (e.g., movement, body muscle, body fat, body hair), sensor degradation, conductivity, environmental conditions, transmission, and the like. The one or more artificial intelligence or machine learning techniques may take any number of actions on the noisy data including regenerating artificial data values as a substitute to noisy values, as well as adjusting noisy values to fit within a preestablished threshold/range of values. Advantageously, one or more artificial intelligence or machine learning techniques, including the training of one or more neural networks, can be utilized to provide information related to a future occurrence, an evaluation or calculation of a probability or odds, a strategy, or a mitigation of risk. In a variation, at least a portion of the one or more readings and/or its one or more derivatives may be used (1) to formulate one or more strategies; (2) to inform one or more users to take one or more actions; (3) as one or more values upon which one or more wagers are placed; (4) to calculate, modify, or evaluate one or more probabilities or odds (5) to create, enhance, or modify one or more products; (6) as one or more data sets or as part of another one or more data sets utilized in one or more simulations, computations, or analyses; (7) within one or more simulations, an output of which directly or indirectly engages with one or more users; (8) as an input in one or more media or promotions; (9) within one or more browser-based games, or (10) to mitigate or prevent one or more risks. Artificial data utilized for one or more of these uses may be generated via one or more simulations. Additional details of a System for Generating Simulated Animal Data And Models are disclosed in U.S. Pat. No. 62/897,064 filed Sep. 6, 2019; the entire disclosure of which is hereby incorporated by reference.

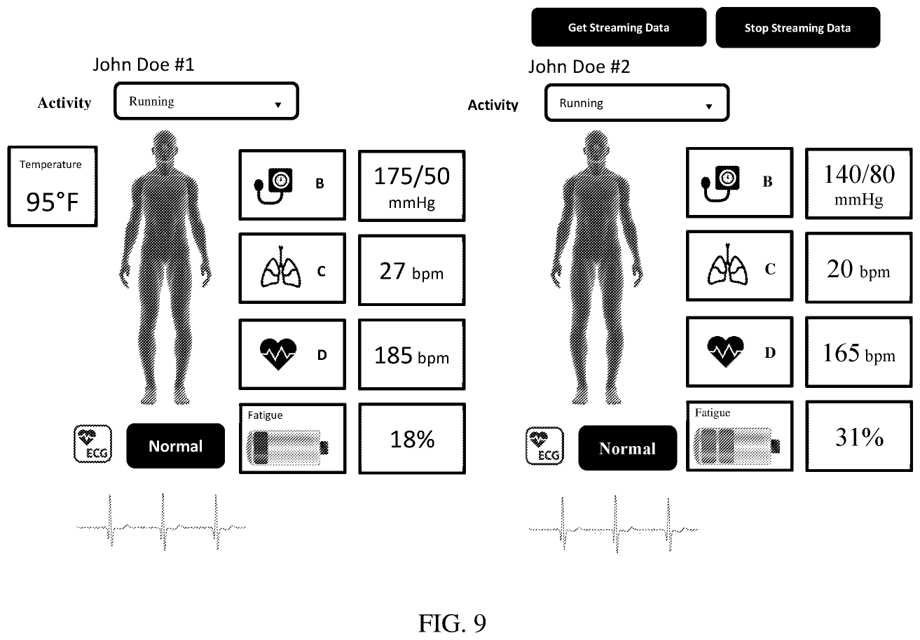

The system operates in a continuous fashion with the connection application receiving one or more data streams continuously or intermittently from the one or more sensors at 60, the connection application sending the one or more data streams to the server at 62, the server processing the one or more data streams to calculate the one or more readings of interest at 64, and the server sending the one or more readings to a web page for display at 66. For example, a data stream may involve electrocardiogram measurements every millisecond and the server may process this stream to calculate a heart rate reading every second. Sensor data sent to a computing device may be either raw or processed depending on the sensor, its capabilities, and/or user preference. In some cases, sensors may have the capability to provide both. FIG. 9 illustrates an exemplary web page for displaying readings. Advantageously, the connection application may be accessed by multiple users simultaneously, so that the information displayed on the web page may be communicated to multiple users at the same time. For example, if a subject is wearing one or more sensors that is sending sensor data to the computing device/server for display, the display and corresponding readings may also be accessed by one or more other users simultaneously, which may occur on one or more separate computing devices. In yet another advantage, the ability to communicate with one or more sensors can occur with any web browser-based system as described herein, with multiple computing devices having the ability to communicate with a single server. For example, a person wearing one or more sensors that communicates with a web-browser based system in their home, a different computing device with a web browser-based system at their fitness facility, and yet a different computing device with a web browser-based system at their office may all communicate with the same sever while utilizing different computing devices, with the data being synchronized across all computing devices. In a refinement, the one or more readings from the one or mom sensors from two or more users may be collected by multiple computing devices and displayed in the connection application with a singular view of the data. For example, in FIG. 9, John Doe #1 may be collecting his data in one location, John Doe #2 may be collecting his data in another location (and using a separate computing device), and a doctor or other medical professional may be aggregating data from both users to visualize at least a portion of the real-time or near-real-time data points in FIG. 9. In a refinement, the one or more readings from the one or more sensors to a web browser-based system can be accessed by one or more third parties in real-time or near real-time, which may occur by accessing the same application which is collecting the sensor data but viewed from one or more different display devices.

Different types of sensors, especially if they am provided by different manufacturers, may employ different communication interfaces. Communication interfaces include command sets, data formats, and message exchange sequences. For example, one type of sensor might begin sending measurements once every 20 milliseconds in response to a single command to begin streaming data. Another type of sensor might require that the measurement frequency be specified in the “begin streaming” command. Yet another type of sensor might send individual measurements in response to request commands such that the system must send requests commands at the desired frequency. In another example, one sensor might encrypt the data and the system receiving the data packets may have to decrypt the data. In another example, one sensor may break large chunks of data into smaller pieces and the receiving system may have to merge smaller chunks to recreate the large data packet. In yet another example, various error codes can be deployed by one or more sensors to indicate error conditions, with each sensor potentially having a different set of error codes or none at all. In yet another example, sensors can use various data formats; for example, one sensor may use hexadecimal (for example), another sensor using binary (for example), while another sensor may use ASCII text.

The web browser-based connection application may be set up to utilize two or more different sensors for a single user. For example, a user may utilize a heart rate sensor and a blood pressure sensor. The web browser-based system may also be set up to utilize two or more sensors on multiple users, which may be communicating with the web browser-based system via a single connection application. For example, two different types of sensors that are communicating with the web browser-based system may be on a plurality of users (e.g., watch-based and chest strap-based heart rate sensors on a plurality of users within a group fitness class that utilizes a web browser-based system). The web browser-based connection application may also utilize two or more sensors that use two or more communication protocols. For example, a user may wear a heart rate monitor that transmits data via BlueTooth and a sweat analysis-based sensor that uses Zigbee. In a refinement, the one or more sensors may have inherent characteristics that enable it to utilize multiple transmission protocols. For example, a sensor may have built-in capabilities that allow for both NFC (passive) and Bluetooth-enabled (active) communication, or both Bluetooth and WiFi communication.

The one or more readings may be displayed and utilized in a number of ways. For example, the readings derived at least in part from one or more sensors may be utilized as part of, or within, a browser-based video game or game-based system (e.g., traditional PC gaming, handheld gaming, virtual reality system, augmented reality system, mixed reality system, and extended reality system, or anything that includes competition or attainment of one or more goals), or a video game or game-based system that accesses the biological data from a browser-based system (e.g., game-based fitness or wellness platform). More specifically, a video game user's readings from one or more sensors may be integrated into a video game or gaming-based system. In one refinement, video game or game-based system hardware may have one or more biological sensors embedded therein (e.g., game controller, game headset, game keyboard, other game sensors), or a user has one or more non-game hardware-based sensors (e.g., smartwatch or on-body sensor capturing biological data) that are in communication with the video game itself via the browser. Examples include hand and finger pressure sensors located within a video game controller (e.g., see how tight the controller is being held), EEG sensors located within a headset utilized as part of a video game, heart rate sensors monitoring the heart rate of the player playing the game, sensors embedded within a bicycle to measure power output or wattage based upon peddle exertion, and reaction time of the player playing the game. In a refinement, the video game or gaming-based system may integrate and display one or more biological readings and/or its one or more derivatives derived from at least a portion of the one or more sensors utilized by one or more users within the browser-based game. For example, participants in a group fitness class may compete with each other in a browser-based game, with one or more of their biological readings being incorporated within the game. In another refinement, the video game or gaming-based system may create one or more new data types for the character or subject within the game based on at least a portion of the real sensor data provided by the user. For example, a browser-based game may create new indices for the in-game subject based on real-world sensor data captured, or insights derived from at least a portion of the sensor data, like fatigue level, heart rate, reaction time, or controller pressure of a mal-world subject. This artificial data utilized within the game, also referred to as simulated data, may be generated by running one or more simulations that utilize at least a portion of the real sensor data to create the artificial data. Providing one or more readings to a video game or game-based system, as well as generating artificial data, can all occur in real-time or near mal-time.

In a variation, one or more users in a web browser-based video game or game-based system can include their own biological data as part of the game and compete against (1) other real-world subjects (e.g., humans that are professional sports athletes, fitness instructors), or (2) virtual participants that may share at least one characteristic with one or more subjects. The system may run one or more simulations to convert real-world biological data into simulated data to be used in the game. For example, a user may want to compete in ahead-to-head tennis match with Athlete X within a web browser-based game, or with a virtual fitness instructor within a web browser-based game, which would include simulated biological data based on at least a portion of the real biological data from both the one or more users and Athlete X. Both the user and Athlete X may utilize one or mom sensors that transmit a variety of biological data such as ECG and heart rate to the web browser-based system, which may be further computed into one or more additional readings (e.g., stress levels) and/or converted into a game-based metric based on one or more simulations (e.g., an “energy level” bar). In a refinement, the one or more game users or spectators participating in or watching the game will have the ability to place a wager based on the game/competition (e.g., on the match played against Athlete X within the gaming system), determine probability or odds for an occurrence of an outcome of an event, revise previously determined probability or odds for an event, or formulate a strategy.

In another refinement, the ability to place one or more wagers/bets on any portion of a video game or game-based system that utilizes at least a portion of the biological sensor data and/or its one or more derivatives occurs via a web browser-based system. In yet another refinement, at least a portion of the sensor data and/or its one or more derivatives, including simulated data, can be provided to a web browser-based system to create one or more new markets (e.g., proposition bets/wagers) for people to place one or more bets. The one or more bets may be based on biological data (e.g., is Player A's heart rate in a live tennis match going to be above 180 bpm in the first set of Match X, which can be a proposition bet offered within a web browser-based system or by a non-web browser-based third party that collects the data from a web browser-based system), or a derivative (e.g., is the character's “energy level” in a soccer video game going to be go below 40% in the first half, with “energy level” being derived from at least a portion of the biological sensor data form a real player or subject and generated from one or more simulations). Simulated data generated from at least a portion of the biological sensor data provided directly to a web browser can also be used to understand the probability of an occurrence for any given outcome and provide predictive insights via one or more simulations. For example, a bettor may have an opportunity to purchase the simulated “energy level” of Player A for the last 10 minutes of a match within a real match or a video game to determine whether Player A will win the match (or win within the video game), with one or more simulations being run to predict the outcome. Artificial data generated from at least a portion of the biological sensor data provided directly to a web browser can also be used to influence the outcome of a particular bet (e.g., by providing an advantage or disadvantage to one or more users within the game) or occurrence within a game. For example, a bettor can purchase more virtual “energy” for virtual player A within the video game to increase the likelihood of Player A winning the game. Additional details related to an Animal Data Prediction System are disclosed in U.S. Pat. No. 62/833,970 filed Apr. 15, 2019 and U.S. Pat. No. 62/912,822 filed on Oct. 9, 2019; the entire disclosures of which are hereby incorporated by reference.

In exchange for providing at least a portion of their biological data, the one or more participants in the game or competition (e.g., Athlete X and/or the one or more game players) may receive a portion of the winnings from any wagers placed or purchases made within the competition that directly or indirectly utilizes their data. For example, in a fitness class, an instructor may be able to receive compensation on any bets made between the instructor and user (e.g., who can pedal the most miles in 10 minutes), or a gamer may receive compensation for proposition bets that incorporate at least a portion of their biological data. In a refinement, one or more subjects that provide at least a portion of their biological data to one or more third parties may receive consideration for providing access to their data. Additional details related to a Monetization System for Human Data are disclosed in U.S. Pat. No. 62/834,131 filed Apr. 15, 2019 and U.S. Pat. No. 62/912,210 iled Oct. 8, 2019; the entire disclosures of which is hereby incorporated by reference.

In a variation, biological sensor data provided directly to a web browser can be utilized to influence one or more outcomes or gain one or more competitive advantages within a gaming system. The data may be provided directly to the gaming system via the web browser or accessed by the gaming system once the data is provided to a web browser-based system. If a gaming system utilizes real-world people or characters that share one or more characteristics of one or more real-world people, the system could utilize at least a portion of the person's real-world sensor data or artificial data, based in part on their real-world biological data, to influence the outcome or provide the ability to influence the outcome through in-game purchases, acquisitions, or achievements of any simulated game played (e.g., sports video game, online virtual world game, group fitness competition). For example, if the user has a high-stress level or has an elevated heart rate (comparative to other users who are also playing a similar game or relative to their baseline heart rate), the one or more subjects within the game may also experience similar data-related responses (e.g., high stress level, elevated heart rate) which may provide an advantage, disadvantage or other indication to the user and/or the one or more subjects in the game. The advantage, disadvantage, or other indication may be immediate and/or for a specific duration. Depending on the game, the advantage may include bonus points, extra strength, access to easier levels of resistance on a bicycle (for example, in a cycling class in which you are competing with other subjects), and the like. Disadvantages within the game may include points lost, a decrease in energy level, more resistance applied to the pedals on a subject's bicycle (for example, in a cycling class in which you are competing with other subjects), and the like. Similarly, the indication of a user's various biological-based animal data readings may include a viewable portal that provides various biological-based animal data readings of the user within the game. Use cases include flight simulations, military simulators, medical simulators, high-frequency trading programs that monitor how traders react in any given situation related to the financial markets, sports video games, fitness classes, everyday wellness monitoring as part of a competition, and the like to understand user behavior and other indicators. As an example, if a user is playing a web browser-based shooting game, demonstrating real stress or an elevated heart rate may make the shooter zoom lens within the game less steady. On the other hand, showing peak biological activity (e.g., steady hands and steady heart rate) may provide the user and their corresponding character or subject in the game (e.g., shooter) with an advantage within the game. These biological data-based animal readings (e.g., real-time heart rate) may be viewed by one or more opponents or third parties, upon which tactics may be created to put the opponent at a disadvantage (e.g., elevate the opponent's real-time heart rate and weaken the opponent in some way within the game), feedback may be provided, a reward or other consideration may be given, etc. In a refinement, the web browser-based system may enable a user of a video game or game-based system to purchase artificial data based upon at least a portion of the sensor data collected by the video game or game-based system. This data may be utilized, for example, to gain an advantage within the game. In the context of a sports video game, the type of artificial data based upon real sensor data that may be purchased within a game may include an ability to run faster, jump higher, have longer energy life, hit the ball farther, or an increase in energy level, which may provide a greater likelihood of winning the game. The type of artificial biological data provided may also include providing one or more special powers to the one or more subjects within the game, with the one or more special powers utilizing at least one related characteristic to the biological data, which may be derived from one or more simulations.

The web browser-based system may also include real-world fitness or wellness applications or programs, including personalized or group fitness classes (e.g., cycling, cross-fit), in which users that am providing at least a portion of their biological sensor data directly to a web browser (e.g., heart rate data) can gain a competitive advantage or other consideration compared to other users within the class (e.g., more rest period, win a free class-based on physiological-based success metrics, win free prizes based on the most “energy” exerted in the class). Comparative biological metrics may be visually displayed for each user via the web browser-based system in order to determine who is performing the best in any given class. In addition, the web browser-based system may be utilized for a wide variety of fitness and personal health/wellness-related opportunities. For example, an application and a transmission subsystem attached to or integrated with hardware of a fitness machine (e.g., treadmill) may communicate with one or more sensors on a subject via a web browser-based system to aggregate all sensor data into a single connection application for real-time or near real-time display of one or more biological metrics. In another example, a gym or fitness studio may provide one or more sensors for its customers in order to monitor and display their real-time or near real-time biological data via a web-browser based system. In another example, a fitness machine with an integrated display may utilize a web browser-based system to collect biological sensor data and create and/or provide one or more biological insights based upon collected biological sensor data to its one or more users during or after a workout (e.g., providing “performance zones” which are based upon a user's minimum and maximum heart rate zones). In a refinement, the fitness machine with computing capabilities may take an action that will adjust the workout based upon the collected biological data (e.g., if the machine determines via the web-browser based system that a user is not working out hard enough or exerting too much energy based on biological data such as heart rate or a derivative such as performance zone, the fitness machine may adjust the difficulty or speed in order to increase or decrease the difficulty of the workout for the user). In a variation, a fitness instructor or “smart” equipment (e.g., equipment with one or more computing capabilities) may take an action that will adjust the workout based upon the collected biological data (e.g., if the instructor or “smart” equipment determines via the web-browser based system that a user is not working out hard enough or exerting too much energy based on biological data such as heart rate or a derivative such as performance zone, the instructor or “smart” equipment may adjust the difficulty or speed in order to increase or decrease the difficulty of the workout for the user). In a refinement, one or more users may receive consideration (e.g., money, gift cards) for allowing a third-party to purchase access to their fitness data. Users may have the ability to opt-in or opt-out prior to, or after, a workout.

In another example, at least a portion of the biological sensor data provided directly to a browser and/or its one or more derivatives (e.g., artificially-generated data, aggregated insights) can be utilized within a wide range of areas including health-monitoring (construction, municipal, oil & gas, hospitals, rehabilitation facilities, general wellness) and personal or group wellness. For example, enabling direct biological sensor communication to a web browser-based system may provide real-time or near-real-time feedback in the confines of one or more subject's homes, in an automobile or autonomous vehicle, in an aircraft, or other areas where a web browser may be accessible. In a refinement, at least a portion of the biological sensor data provided directly to a web browser and/or its one or more derivatives (e.g., simulated data) is provided to, or accessed by, a third party and/or multiple parties. For example, one or more subjects may choose to send at least a portion of their biological sensor data to a third-party medical or health-related organization directly (e.g., doctor, hospital, insurance) via a web browser-based system to obtain real-time or near real-time feedback related to the sensor data. Medical professionals may access real-time information via a web browser remotely from one or more patients that are in different locations. The information may be provided to a web browser-based portal controlled by a third party (e.g., the doctor or hospital) or by the individual (which is then accessed by the medical professional or hospital). In another example, one or more subjects may choose to send at least a portion of their biological sensor data to an insurance company directly via a web browser-based system during a physical or other medical-related examination to have a premium adjusted and/or receive a benefit. In yet another example, one or more subjects may choose to send at least a portion of their biological sensor data to a third-party analytics company via a web browser to be able to create one or more insights derived from at least a portion of the biological sensor data for the benefit of themselves (e.g., receiving feedback about their own health status via the web browser or other application) or a third party (e.g., providing the biological sensor data directly via a web browser to a pharmaceutical company that is monitoring the effects that a particular drug may have on any given subject).

Advantageously, the biological sensor data collected by the web browser may be accessed simultaneously by one or more parties in different locations and/or from multiple computing devices. For example, a web browser-based system may be utilized in a medical or clinical setting whereby the system is collecting the patient's real-time vitals while being accessible by doctors or other medical professionals in multiple locations and/or from multiple computing devices via the web browser. In another example, a military organization may want to access the real-time vitals of its soldiers from multiple locations and/or from-multiple computing devices. In another example, a fitness instructor or fitness organization may want to access the real-time vitals of its one or more users from multiple locations and/or from multiple computing devices. In another example, a sports team may want to access the real-time biological data of its athletes from multiple locations within a venue via the web. In yet another example, a sports betting application may provide real-time biological data as a service to its paying customers that are accessing the data from multiple locations and/or from multiple computing devices.

In a refinement, different functionalities performed within the connection application may be executed on separate computing devices and/or within multiple browsers. For example, a doctor or other medical professional may access a patient's profile within a web browser executing the connection application and change, adjust, or modify one or more sensor settings while the user is streaming data from one or more sensors to the web browser-based connection application. Advantageously, this may occur via access to the connection application in a separate web browser, or in a separate connection application utilized by the doctor that communicates with the web-browser based application. Communication may not necessarily need to be real-time and can occur at different times. For example, a doctor or other medical professional may go into the connection application to change a setting for a patient (e.g., a dosage of a specific drug administrated by a sensor, a sampling rate of a specific sensor, a firmware update). When the patient loads the connection application into the web browser, which may occur at a later time, the changed setting will be communicated to the sensor, and the setting will be changed. In a refinement, the doctor or other medical professional may take control of the user's connection application, either via a web browser or other mechanism to control the computing device (e.g., screen share, other application) and change the one or more settings on the user's browser-based connection application. For example, if a medical professional is monitoring an athlete on a treadmill remotely and the athlete has multiple sensors on his body, the medical professional (e.g., doctor) may adjust the settings on her/his computing device via a mechanism that enables control of the athlete's web-browser based computing device, providing the doctor with access to the connection application remotely. This may occur in a separate browser (e.g., if the doctor is using a browser), within the same browser (if using a screen share function or other control that enables the doctor to access the athlete's web browser), or using another connection application that communicates with the browser-based connection application utilized by the athlete (e.g., a non-browser based application that enables control of the browser-based connection application).

In a refinement, one or more users can access at least a portion of the one or more streamed data readings and/or its one or more derivatives via two or more computing devices. For example, a patient may stream physiological data readings (e.g., heart rate, blood pressure) from one or more sensors worn by the patient to the web browser-based connection application, which is also accessed by her/his medical professional that is viewing the same information but on a separate computing device. This information may be viewed by the medical professional simultaneously as the data is being streamed to the connection application (e.g., in real-time), in near real-time, or at a later date (e.g., the patient streams the data to the system in the morning, and the medical professional views the data in the afternoon). Advantageously, this information may also be viewed by one or more third parties (e.g., the doctor) in a non-browser-based connection application.

In another refinement, one or more actions are initiated or programmed to occur within the browser application via two or more computing devices, at least one of which utilizes the browser application and receives a portion of the streamed data from the one or more sensors. For example, a patient may take an action to add a sensor or stream their physiological data readings (e.g., heart rate, blood pressure) from one or more sensors to the web browser-based connection application, while the doctor or other medical professional may take a separate action (e.g., change one or more of the sensor settings or functionality) on a separate computing device within the browser-based connection application. Different functionalities that are initiated or programmed to occur within the browser-based connection application can also occur utilizing different platforms. Advantageously, the doctor or other medical professional may be able to change the one or more sensor settings (or take any action) in a non-browser-based connection application, which would communicate with the browser-based connection application to take the one or more actions. The web browser-based connection application could also be set up like a Master/Slave communication process whereby the patient's web browser-based connection application (Slave) is controlled by the doctor/medical professional or other third party (Master), which may be a web browser-based connection application, a non-web browser-based connection application, or other control mechanism. Depending on the setup, the patient may need to take one or more steps to accept the doctor/medical professional's (or third party) changes.

In another refinement, one or more commands are initiated on at least one computing device that is not in direct communication with the one or more sensors. For example, one or more sensor settings/parameters/functionalities can be changed, adjusted, or modified within the browser-based connection application through a computing device that is not in direct communication with the one or more sensors. This may occur via the connection application, a separate application, or other control mechanisms. In a variation, the computing device providing the one or more commands may not be programmed to directly communicate with the one or more sensors. In another variation, one or more users access the connection application within a browser from two or more computing devices simultaneously, with one or more computing devices programmed to control, change, and/or modify one or more sensor settings within the one or more browser-based connection applications on the one or more other computing devices. For example, the web browser-based system may include research and clinical applications or programs, where there is a need to control an entire program (or partial scope) for a group of individuals. Similar to a Master/Slave setup, a full program may consist of the web browser of the “parent” computing device that controls all web browser settings and functions of the “children” computing devices and its associated sensors for use during a pre-defined session or multiple sessions over a set period of time (e.g., week, month, year). For example, the ability for a “parent” to control the functions and settings of the “children” web browser, computing device and associated sensors enables a research or clinical study to dictate the specifics of the program in real-time and adjust things as needed instantly and provides a level of control over a subject group. In a refinement, control, change, and/or modification of the one or more sensor settings within the connection application via the one or more browsers is programmed to occur for a single user, a subset of users, and/or all users of the connection application. For example, an administrator of a program may only want to adjust the sensor settings on a subset of users within their web browser-based application while not adjusting the settings for another group.

While multiple users wearing one or more sensors can stream their data to a single connection application within a single web browser, another advantage of the system is that two or more sensors can communicate with the connection application simultaneously through multiple computing devices or multiple browsers. For example, in a remote fitness class featuring 20 participants, each user may be streaming one or more physiological parameters (e.g., heart rate, respiration rate) to the browser-based connection application on each of the 20 individual computing devices, which may then be viewed in real-time and simultaneously by the fitness coach through the browser-based connection application.

In another refinement, one or more sensors communicate with the connection application via a plurality of browsers or computing devices. In many use cases, communication does not occur at the same time and occurs in succession and/or intermittently. For example, an organization that operates a multitude of equipment (e.g., trucks, cranes, farm equipment) that includes a computing device and programmed to employ multiple, interchangeable individuals to operate each equipment may want to continuously monitor one or more data readings of each individual, a subset of individuals, or all the individuals as they operate each equipment, regardless of which equipment they are operating. By utilizing a web browser-based communication on or nearby the equipment with one or more on each of the individuals, and potentially on other subjects (e.g., the machinery/equipment) that are in communication with the connection application via a plurality of web browsers or computing devices, the system will enable seamless data collection for the organization and its employees. The system may be a single computing device comprised of multiple hardware components that display the connection application via a web browser and communicate with the one or more sensors, or multiple computing devices, each with their own ability to display the connection application via the web browser. In a refinement, an organization or individuals within an organization with multiple employees in multiple locations can have alerts and notifications sent to a variety of agencies (e.g., corporate, fire department, hospital, paramedics, emergency contacts) in the event that any given subject's reading reaches a pre-defined threshold that would trigger such an alert (e.g., heart rate over a pre-defined threshold for a pre-defined period of time for a specific activity).

In addition to being operable to communicate with biological sensors, the connection application may also be programmed to transmit one or more commands to one or more wireless communication-enabled sensors that output non-biological data, with the one or more non-biological sensors programmed to take one or more actions based on the one or more commands. A non-biological sensor could provide video data (e.g., a webcam or camera providing a video and audio feed of one or more users) from which no biological data can be extracted, environmental data (e.g., humidity, temperature), auditory data from which no biological data can be extracted, data derived from non-subjects such as force, pressure, fluid level, flow, and ambient light, and the like. Sensors that output non-biological data may or may not be wearable, can take a variety of forms, and could include any form of hardware with a computing device including AR/VR systems, machines, robots, and the like. Advantageously, the one or more non-biological sensors, computing device, transmission subsystem, server, and the display device, or a combination thereof, may be housed within, attached to, or comprise the same unit. In a refinement, the connection application is operable to receive at least a portion of non-biological data from one or more sensors, with the data being displayed, at least in part, within the browser-based connection application.

FIG. 10 illustrates an exemplary display page for demonstrating both an ability to provide a sensor outputting non-biological data with a command (and the sensor taking an action based upon the command), as well as an ability to receive and display the streamed data within the browser-based connection application from a non-biological sensor. In this example, a user (e.g., doctor or other medical professionals) of the connection application may want to view (via video) another user of the connection application (e.g., a patient) who is also wearing a biological sensor. The video provided by the webcam, which is data comprised of images and audio files that are digitally transmitted, is received by the connection application and rendering for the doctor or other medical professionals to see. A medical professional or other users may, for example, send one or more commands to the camera (e.g., turn on) prior to seeing the user, or send one or more commands (e.g., maneuver the camera to look at different parts of the body to sure the one or more sensors are on correctly) using functionality 90 while viewing the patient and their one or more biological readings. Advantageously, the doctor or other medical professional may utilize a different window or terminal within the connection application to control, adjust, and/or modify any given sensor setting. As in previous examples, the display may not be visual. In the case of an audio sensor, the display within the browser-based application may be a streamed audio file.

In another example, a manager of the research or clinical study may have the ability to view one or all of the subjects via a camera-based video feed as well as all of their metrics and settings for each sensor. The camera may be integrated into the computing device being used to display the browser-based application or may be a separate component. The video may be real-time or may be captured and recorded for viewing at a later time. The one or more subjects may also have the ability to view the manager (or administrator) for two-way video communication. In another example, a doctor or other medical professional may provide a command to a robotic arm directly via the browser-based connection application in order to perform a specific task (e.g., for the arm to move or take an action during surgery) while the patient's biological readings are being monitored, or an operator of a manufacturing plant may provide one or more commands to a machine to perform a specific task from a remote location via the browser-based connection application. In another example, a command may be provided from a browser-based connection application for a robot or machine to take one or more actions on behalf of a subject.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. As such, embodiments described as less desirable than other embodiments or prior art implementations with respect to one or more characteristics are not outside the scope of the disclosure and can be desirable for particular applications.

What is claimed is:

1. A biological data tracking system comprising:
one or more wireless communication-enabled data sensors;
a computing device including a network connection and a browser application; and
a connection application executing within the browser application, the connection application configured to establish one or more wireless communication links with each of the one or more wireless communication-enabled data sensors, receive one or more data streams from the one or more wireless communication-enabled data sensors, and display, via the browser application, one or more data readings derived from, at least in part, the streamed data.

2. The biological data tracking system of claim 1 further comprising a server configured to receive at least a portion of the one or more data streams via the network connection and compute the one or more data readings.

3. The biological data tracking system of claim 2 wherein the server is further configured to transmit the connection application to the browser application in response to a user accessing a web page.

4. The biological data tracking system in claim 1 wherein one or more users access at least a portion of the one or more data streams, the one or more data readings, and/or its one or more derivatives via two or more computing devices.

5. The biological data tracking system in claim 1 wherein one or more actions are initiated or programmed to occur within the browser application via two or more computing devices, at least one of which utilizes the browser application and is operable to receive at least a portion of the one or more data streams from the one or more wireless communication-enabled data sensors.

6. The biological data tracking system of claim 1 wherein the one or more wireless communication-enabled data sensors includes at least two different types of sensors.

7. The biological data tracking system of claim 6 wherein the at least two different types of sensors communicate with the connection application using different communication interfaces.

8. The biological data tracking system of claim 6 wherein at least one reading of the one or more data readings is derived from one or more data streams from the at least two different types of sensors.

9. The biological data tracking system in claim 1 wherein at least a portion of the one or more data readings and/or its one or more derivatives are generated or adjusted, at least in part, by one or more artificial intelligence or machine learning techniques.

10. The biological data tracking system in claim 1 wherein at least a portion of the one or more data readings and/or its one or more derivatives are used (1) to formulate one or more strategies; (2) to inform one or more users to take one or more actions; (3) as one or more values upon which one or more wagers are placed; (4) to calculate, modify, or evaluate one or more probabilities or odds; (5) to create, enhance, or modify one or more products; (6) as one or more data sets or as part of another one or more data sets utilized in one or more simulations, computations, or analyses; (7) within one or more simulations, an output of which directly or indirectly engages with one or more users; (8) as an input in one or more media or promotions; (9) within one or more browser-based games; or (10) to mitigate or prevent one or more risks.

11. The biological data tracking system of claim 1 wherein the connection application is further programmed to transmit one or more commands to a remote-controlled device and/or to the one or more wireless communication-enabled data sensors to change, adjust, and/or modify one or more sensor settings.

12. The biological data tracking system of claim 11 wherein the one or more commands change, adjust, and/or modify administration of one or more substances.

13. The biological data tracking system of claim 12 wherein administration of one or more substances is determined, at least in part, via one or more calculations, computations, predictions, estimations, inferences, observations, or forecasts that that utilize at least a portion of one or more data readings and/or its one or more derivatives.

14. The biological data tracking system in claim 11 wherein the one or more commands are initiated on at least one computing device that is not in direct communication with the one or more wireless communication-enabled data sensors.

15. The biological data tracking system in claim 1 wherein two or more sensors communicate with the connection application simultaneously through multiple computing devices and/or multiple browsers.

16. The biological data tracking system of claim 1 wherein the one or more wireless communication-enabled data sensors communicate with the connection application via a plurality of browsers and/or computing devices.

17. The biological data tracking system in claim 1 wherein one or more users access the connection application from two or more computing devices simultaneously, with one or more computing devices programmed to change, adjust, and/or modify one or more sensor settings within the connection application on other computing devices.

18. The biological data tracking system in claim 17 wherein change, adjustment, and/or modification of one or more of the sensor settings within the connection application via one or more browsers occurs for a single user, a subset of users, and/or all users.

19. The biological data tracking system in claim 1 wherein the connection application is further programmed to transmit one or more commands to one or more sensors that output at least a portion of non-biological data, with the one or more sensors programmed to take one or more actions based on the one or more commands provided by the connection application.

20. The biological data tracking system in claim 1 wherein the connection application is further programmed to receive at least a portion of non-biological data from one or more sensors, with data being displayed, at least in part, within the browser application.

21. The biological data tracking system of claim 1 wherein the connection application is further configured to establish one or more communication links with one or more wired sensors.

22. A biological data tracking system comprising:
a connection application, executable within a web browser, programmed to establish one or more wireless communication links with a plurality of wireless communication-enabled sensors, receive one or more data streams from the plurality wireless communication-enabled data sensors, and display one or more readings derived from, at least in part, the one or more data streams; and
a server configured to transmit the connection application to the web browser in response to a user accessing a web page.

23. The biological data tracking system of claim 22 wherein:
the connection application transmits the one or more streams of data to the server; and
the server calculates the one or more readings.

24. The biological data tracking system of claim 22 wherein the plurality of wireless communication-enabled sensors includes at least two different types of sensors.

25. The biological data tracking system of claim 24 wherein the two different types of sensors communicate with the connection application using different communication interfaces.

26. The biological data tracking system of claim 22 wherein at least one reading of the readings is derived from one or more data streams from two or more different sensors.

27. The biological data tracking system of claim 22 wherein the connection application is further programmed to transmit one or more commands to a subset of the plurality of wireless communication-enabled sensors to change, adjust, and/or modify one or more sensor settings.

28. A method of tracking biological data comprising:
transmitting a connection application from a server to a browser in response to a user accessing a web page;
detecting, via the connection application, one or more wireless communication-enabled sensors;
connecting, via the connection application, to the one or more wireless communication-enabled sensors
wirelessly receiving, via the connection application, one or more data streams from each of the one or more wireless communication-enabled sensors; and
displaying, within the browser, one or more readings derived from at least a portion of one of the data streams.

29. The method of claim 28 further comprising:
transmitting the one or more data streams from the browser to the server; and
transmitting the one or more readings from the server to the browser.

30. The method of claim 28 further comprising sending one or more commands from the connection application to the one or more wireless communication-enabled sensors to change, adjust, and/or modify at least one sensor setting.

31. The method of claim 28 wherein the one or more wireless communication-enabled sensors includes at least two different types of sensors.

32. The method of claim 31 wherein the at least two different types of sensors transmit one or more data streams to the connection application using at least two different communication interfaces.

33. The method of claim 28 wherein the one or more readings are derived from one or more data streams from more than one sensor.
